(19) United States
(12) Patent Application Publication
(10) Pub. No.: US 2009/0082847 A1
(43) Pub. Date: Mar. 26, 2009

(54) SYSTEM AND METHOD OF SECURING STENT BARBS

(75) Inventors: Isaac J. Zacharias, Pleasanton, CA (US); Maurice Marthaler, Santa Rosa, CA (US); Chris L. Staudenmayer, Santa Rosa, CA (US); Brian A. Glynn, Santa Rosa, CA (US)

Correspondence Address: GRANT ANDERSON LLP, GRANT ANDERSON LLP C/O PORTFOLIOIP, P.O. BOX 52050, MINNEAPOLIS, MN, 55402, US

(73) Assignee: BOSTON SCIENTIFIC CORPORATION, Maple Grove, MN (US)

(21) Appl. No.: 11/861,731

(22) Filed: Sep. 26, 2007

(51) Int. Cl. A61F 2/82

(52) U.S. Cl. 623/1.15

(57) ABSTRACT

A stent assembly comprising a stent body. At least one barb extends from the stent body and is configured such that a free end thereof is biased to extend radially outward from the stent body. A belt is releasably positioned about the stent body and aligned with the barb to constrain the barb to a position with the free end proximate to the stent body. A method of forming a stent assembly is also provided.

### BACKGROUND OF THE INVENTION

This invention relates generally to endoluminal devices, particularly stents and grafts for placement in an area of a body lumen that has been weakened by damage or disease, such as an aneurysm of the abdominal aorta, and more particularly to devices having characteristics that enhance affixation of the devices to the body lumen.

Medical devices for placement in a human or other animal body are well known in the art. One class of medical devices comprises endoluminal devices such as stents, stent-grafts, filters, coils, occlusion baskets, valves, and the like. A stent typically is an elongated device used to support an intraluminal wall. In the case of a stenosis, for example, a stent provides an unobstructed conduit through a body lumen in the area of the stenosis. Such a stent may also have a prosthetic graft layer of fabric or covering lining the inside and/or outside thereof. A covered stent is commonly referred to in the art as an intraluminal prosthesis, an endoluminal or endovascular graft (EVG), a stent-graft, or endograft.

An endograft may be used, for example, to treat a vascular aneurysm by removing or reducing the pressure on a weakened part of an artery so as to reduce the risk of rupture. Typically, an endograft is implanted in a blood vessel at the site of a stenosis or aneurysm endoluminally, i.e. by so-called “minimally invasive techniques” in which the endograft, typically restrained in a radially compressed configuration by a sheath, crocheted or knit web, catheter or other means, is delivered by an endograft delivery system or “introducer” to the site where it is required. The introducer may enter the vessel or lumen from an access location outside the body, such as purcutaneously through the patient's skin, or by a “cut down” technique in which the entry vessel or lumen is exposed by minor surgical means. The term “proximal” as used herein refers to portions of the endograft, stent or delivery system relatively closer to the end outside of the body, whereas the term “distal” is used to refer to portions relatively closer to the end inside the body.

After the introducer is advanced into the body lumen to the endograft deployment location, the introducer is manipulated to cause the endograft to be deployed from its constrained configuration, whereupon the stent is expanded to a predetermined diameter at the deployment location, and the introducer is withdrawn. Stent expansion typically is effected by spring elasticity, balloon expansion, and/or by the self-expansion of a thermally or stress-induced return of a memory material to a pre-conditioned expanded configuration.

Among the many applications for endografts is that of deployment in lumen for repair of an aneurysm, such as a thorasic aortic aneurysm (TAA) or an abdominal aortic aneurysm (AAA). An AAA is an area of increased aortic diameter that generally extends from just below the renal arteries to the aortic bifurcation and a TAA most often occurs in the descending thoracic aorta. AAA and TAA generally result from deterioration of the arterial wall, causing a decrease in the structural and elastic properties of the artery. In addition to a loss of elasticity, this deterioration also causes a slow and continuous dilation of the lumen.

The standard surgical repair of AAA or TAA is an extensive and invasive procedure typically requiring a week long hospital stay and an extended recovery period. To avoid the complications of the surgical procedure, practitioners commonly resort to a minimally invasive procedure using an endoluminal endograft to reinforce the weakened vessel wall, as mentioned above. At the site of the aneurysm, the practitioner deploys the endograft, anchoring it above and below the aneurysm to relatively healthy tissue. The anchored endograft diverts blood flow away from the weakened arterial wall, minimizing the exposure of the aneurysm to high pressure.

Intraluminal stents for repairing a damaged or diseased artery or to be used in conjunction with a graft for delivery to an area of a body lumen that has been weakened by disease or damaged, such as an aneurysm of the thorasic or abdominal aorta, are well established in the art of medical science. Intraluminal stents having barbs, hooks, or other affixation means to secure the stents to the wall of the lumen in which they are to be deployed are also well known in the art.

While barbed and the like stents are advantageous in anchoring the device, an improved system for retaining and releasing stent barbs is desired.

### SUMMARY OF THE INVENTION

In one aspect, the invention provides a stent assembly comprising a stent body. At least one barb extends from the stent body and is configured such that a free end thereof is biased to extend radially outward from the stent body. A belt is releasably positioned about the stent body and aligned with the barb to constrain the barb to a position with the free end proximal to the stent body.

In another aspect, the invention provides a method of forming a stent assembly, comprising: forming a stent body having at least one barb with a free end extending radially outward from the stent body; and releasably securing a belt about the stent body in alignment with the barb to constrain the barb to a position with the free end proximate to the stent body.

Other aspects and advantages of the present invention will be apparent from the detailed description of the invention provided hereinafter.

### BRIEF DESCRIPTION OF THE DRAWINGS

The invention is best understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 is a side elevation of a compressed stent with belted barbs in accordance with a first embodiment of the present invention.

FIG. 2 is a side elevation of the compressed stent of FIG. 1 with the barbs released.

FIG. 3 shows a flat pattern of the stent of FIG. 1 illustrating the grinding pattern of the grooves.

FIG. 4 is a side elevation of a compressed stent with belted barbs in accordance with an alternative embodiment of the present invention.

FIG. 5 is a side elevation of the compressed stent of FIG. 4 with the barbs released.

FIG. 6 shows a flat pattern of the stent of FIG. 4 illustrating the grinding pattern of the grooves.

FIG. 7 is a cross-sectional view of a grinding rod of a first method for grinding the stent of FIG. 4.

FIG. 8 is a cross-sectional view similar to FIG. 7 and illustrating a stent positioned on the grinding rod for grinding.

FIG. 9 is an isometric view of an alternative grinding rod and associated collar.

FIG. 10 is a cross-sectional view of the grinding rod of FIG. 9.

FIG. 11 is an end elevation view of the collar of FIG. 9.

FIG. 12 is a cross-sectional view along the line 12-12 in FIG. 11.

FIG. 13 is a side elevation of a compressed stent with belted barbs in accordance with another alternative embodiment of the present invention.

FIG. 14 is a side elevation of the compressed stent of FIG. 13 with the barbs released.

FIG. 15 shows a flat pattern of the stent of FIG. 13 illustrating the grinding pattern of the grooves.

FIG. 16 shows a flat pattern of another alternative stent illustrating the grinding pattern of the grooves.

FIG. 17 is a cross-sectional view of a grinding rod of a method for grinding the stent of FIG. 16.

FIG. 18 is a cross-sectional view similar to FIG. 17 and illustrating a stent positioned on the grinding rod for grinding.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

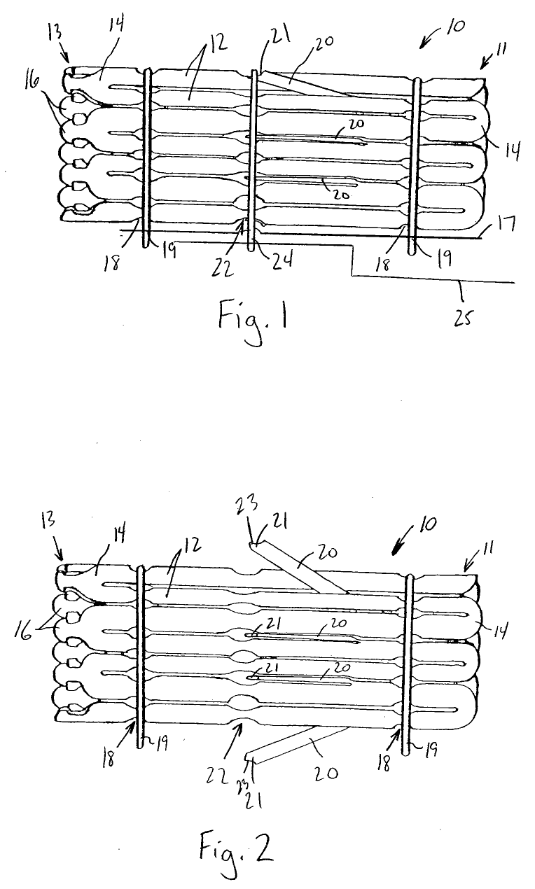

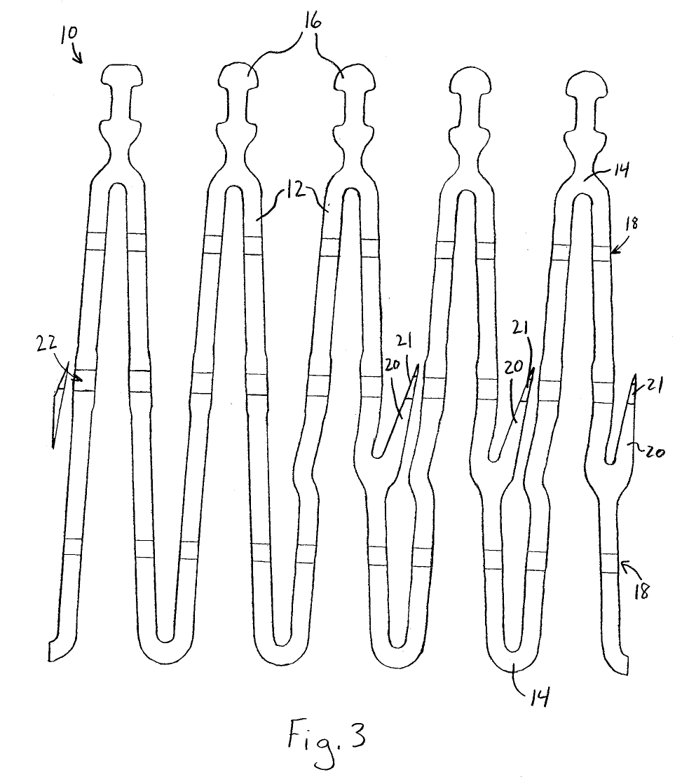

Referring to FIGS. 1-3, a stent 10 that is a first embodiment of the present invention is illustrated, with FIGS. 1 and 2 illustrating the stent 10 schematically and FIG. 3 illustrating a flat pattern of the stent 10. Stent 10 includes a plurality of struts 12 extending axially between the opposed ends 11, 13 thereof. The stent 10 can be oriented in either direction, that is, the end 13 may represent the proximal end or the distal end of the stent 10, depending on the application. Both ends 11, 13 have a plurality of crowns adjoining adjacent struts 12. The end 13 of stent 10 has a plurality of connecting members 16 configured to connect the stent 10 to a graft or other structure. The illustrated stent 10 structure is merely a representative example, and the invention is not intended to be limited to such. The stent 10 of the present invention can have various structures and is not limited to the strut structure illustrated herein. For example, the stent may have a body defined by a lattice structure or a helical structure.

Along one or more of the struts 12, a barb 20 is provided. Referring to FIG. 3, the barbs 20 are preferably formed integrally with the struts 12, but may otherwise be manufactured, for example, as a separate component attached to the struts 12. Each of the barbs 20 has a pointed tip 21 configured to engage the intended lumen wall. In the present embodiment, each tip 21 slopes outwardly along its outward radial extent. The stent struts 12 and the barbs 20 are preferably self expanding, that is, upon release of a constraining force, the struts 12 will move radially apart and the barbs 20 will extend radially outward. Other configurations, for example, balloon expansion, are also contemplated within the present invention.

Referring to FIG. 1, a belt 24 is compressed about the stent 10 and contacts approximately the tips 21 of the barbs 20 to constrain the barbs 20. A release wire 25 or the like preferably extends through the ends of the belt 24 to retain the belt 24 in the constraining condition. The release wire 25 may extend through the barb belt 24 alone with a separate wire 17 extending through the main belts 19 retaining the stent 10, as illustrated. Alternatively, a single wire may pass through all of the belts 19 and 24 and control deployment of the stent 10 and the barbs 20. The belts 19 and 24 and release wires 17 and 25 can be selected to provide various deployment sequences. For example, the barbs 20 may be deployed first, as illustrated in FIG. 2, and thereafter the stent 10 deployed such that the barbs 20 are positioned for engagement as soon as the stent is released. As another example, all of the belts 19 and 24 may be release substantially simultaneously such that the stent 10 opens in a uniform manner. Alternatively, a single belt may be utilized for both maintaining the stent 10 in the compressed configuration and retaining the barbs 20 in the constrained condition. Various belt and release wire configurations and sequences are described in U.S. Patent Application Publication No. US 2004/0138734, which is incorporated herein in its entirety by reference.

To minimize axial movement of the belt 24, a circumferential groove 22 is preferably ground, etched (e.g. laser or chemical) or otherwise formed about the stent 10 axially aligned with the barbs 20. The groove is similar to the circumferential grooves 18 provided for the main belts 19. In the present embodiment, the groove 22 is substantially aligned with the barb tips 21, such that the barb tips 21 have a minimal groove 23 therein. The barbs 20 continue to present a sharpened tip and the groove 23 generally does not affect the barb 20 effectiveness. The groove 22 extending across each of the struts 12 and the barb tips 21 can be seen in the schematic drawing in FIG. 3.

As illustrated in FIG. 1, the belt 24 retains each of the barbs 20 in a constrained position with the sharpest portion of the tip positioned radially inward from the surface of the stent 10, thereby providing effective barb 20 constraint. Additionally, since the barbs 20 are not tucked under the struts 12 or tucking pads (which may be used in prior art devices, but not required with the present device), the barbs 20 are free to reliably expand as soon as the belt 24 is removed. As an additional advantage, since the barbs 20 are not tucked under the struts 12, the stent 10 maintains a slim and more uniform radial profile in the compressed state. In contrast, stents with tucked barbs often have an expanded mid-section, similar to a football shape, due to the double material thickness of the strut and barb tucked underneath.

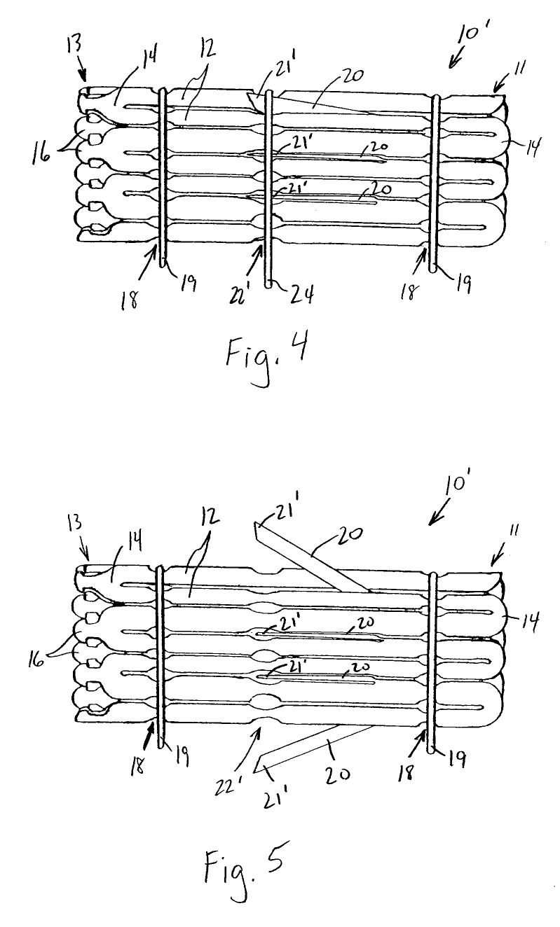

Referring to FIGS. 4-6, a stent 10′ that is an alternative embodiment of the present invention is shown. The stent 10′ is similar to that of the previous embodiment, except that a groove 23 is not present on the barb tips 21′. This is illustrated more clearly in FIG. 6. Referring to FIG. 5, the barb 20 includes a full outwardly directed tip 21′. In addition to providing more material (since there is no groove 23), the belt 24 has the higher radially outward surface of the barb 20 to contact. As such, the belt 24 more effectively depresses the barbs 20 below the outer radial surface of the compressed stent 10′.

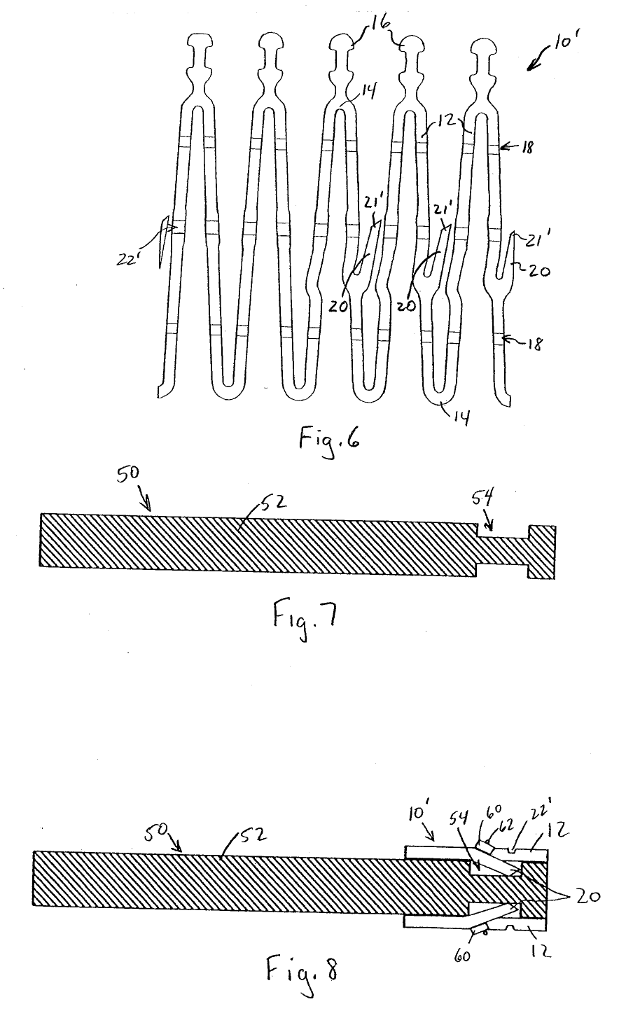

Referring to FIGS. 7 and 8, a first method of manufacturing the stent 10′ of FIGS. 4-6 will be described. A grinding rod 50 has a generally cylindrical body 52 with a circumferential recess 54 formed adjacent one end of the rod 50. The circumferential recess 54 is configured to receive the barbs 20 in an inwardly deflected position such that the barb outer surfaces are below the plane of the grinding wheel (not shown). As shown in FIG. 8, the stent 10′ is positioned on the grinding rod 50 with the barbs 20 axially aligned with the circumferential recess 54. A deflecting block 60 or the like is attached to the outer surface of each barb 20. A wire 62 or the like is then tightened about the deflecting blocks 60 such that the blocks 60, and thereby the barbs 20, are deflected inward. With the barbs 20 deflected into the circumferential groove 54, the grinding wheel can be utilized to grind the barb belt groove 22′. Upon removal of the stent 10′ from the grinding rod 50, the stent struts 12 include the groove 22′, but the barbs 20 do not have the groove 22′, as illustrated schematically in FIG. 6. The main belt grooves 18 may also be ground prior to removal of the stent 10′ from the grinding rod 50.

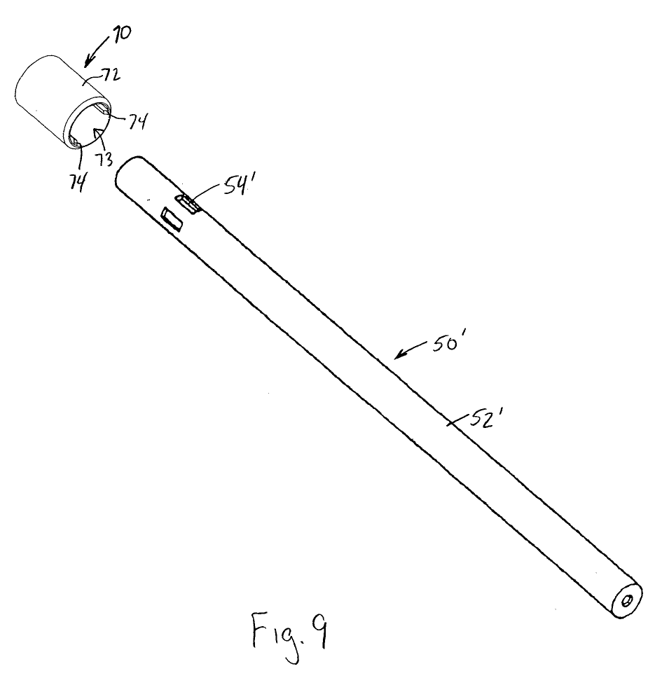

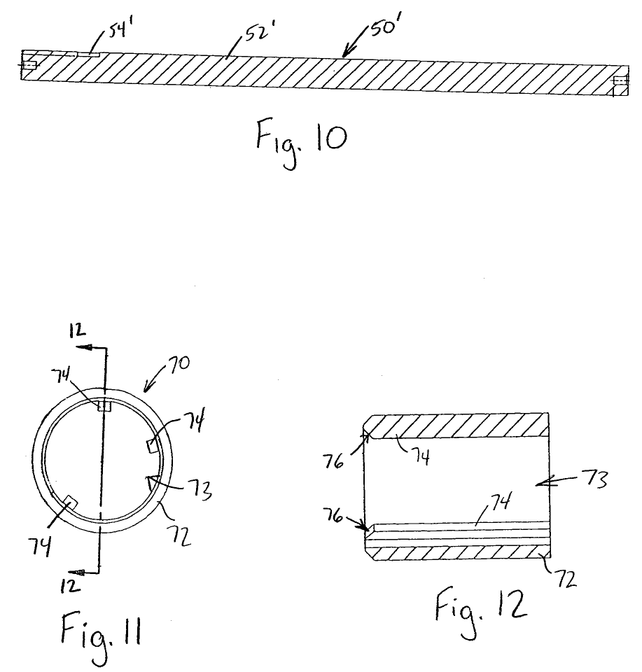

Referring to FIGS. 9-12, an alternative method of manufacturing the stent 10′ of FIGS. 4-6 will be described. The method again utilizes a grinding rod 50′ having a cylindrical body 52′. Instead of providing a full circumferential groove, individual barb slots 54′ are provided in the grinding rod 50′. As such, the barbs 20 can be deflected into the slots 54′ while the struts 12 remain supported along the rod body 52′ during grinding of the groove 22′. To deflect the barbs 20 into the slots 54′, a collar 70 is utilized. The collar 70 includes a cylindrical body 72 with an axial through bore 73 larger than the outer diameter of the stent 10′ when it is positioned on the rod 50′. The collar 70 includes a plurality of inwardly extending ribs 74 corresponding to the number of barbs 20 and slots 54′. The ribs 74 define an inner diameter therebetween which is only slightly larger than the outer diameter of the grinding rod 50′. As such, as the collar 70 is moved onto the grinding rod 50′, the stent struts 12 fit between the collar body 72 and the grinding rod 50′, however, the clearance at the ribs 74 is not sufficient, and the ribs 74 contact the corresponding barbs 20 and deflect the barbs 20 into the corresponding slots 54′. Each of the ribs 74 preferably has a tapered forward end 76 to further facilitate passage of the rib 74 onto the respective barb 20.

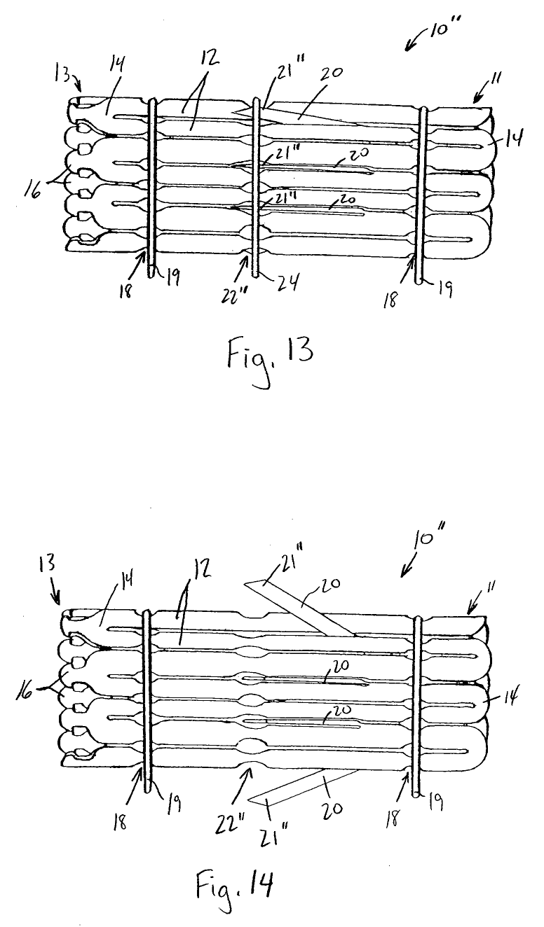

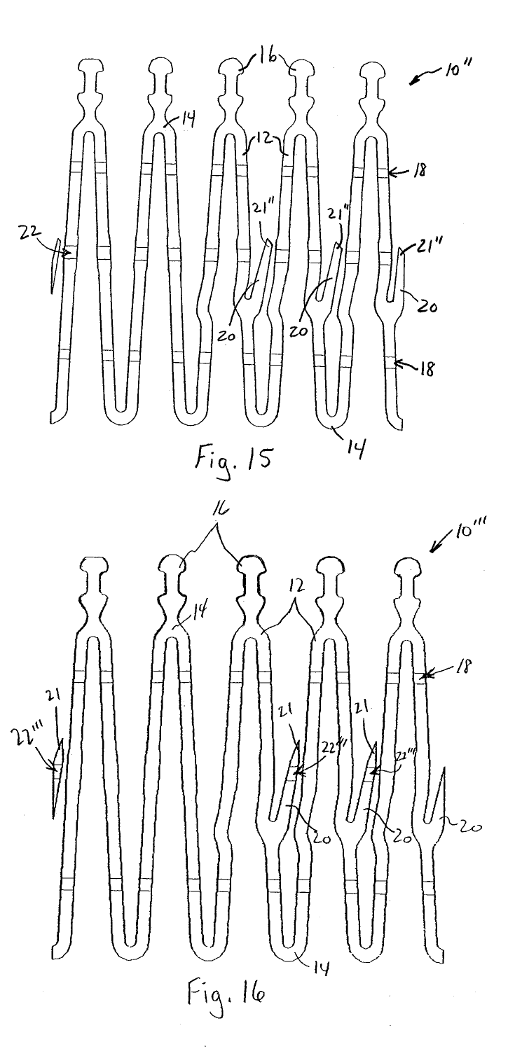

Referring to FIGS. 13-15, a stent 10″ that is an alternative embodiment of the present invention is shown. The stent 10″ is similar to that of the stent 10′ of FIGS. 4-6 and again does not include a groove 22″ extending across the barb tips 21″, as seen in FIG. 15. Referring to FIG. 14, the stent 10″ differs from the stent 10′ in that the barb 20 converges inward to a radially inward tip 21″. As such, the barb 21″ is yet further recessed from the stent outer surface, as illustrate in FIG. 13. In some applications, the inward tip 21″ may also prove more effective since the tip 21″ will effectively lock against radially inward disengagement once it engages the lumen wall.

Referring to FIG. 16, a flat schematic pattern of another alternative stent 10′″ is shown. The current stent 10′″ is in opposite to the stent 10′ of FIGS. 4-6 in that the stent 10′″ includes a belt groove 22′″ extending across the barbs 20, but no associated belt groove extending across the stent struts 12. Such a configuration has been found in some applications to provide a better combination of barb recessing and barb constraining effectiveness.

While various configurations of barb tips are illustrated and described, the invention is not limited to such and other configurations may be utilized.

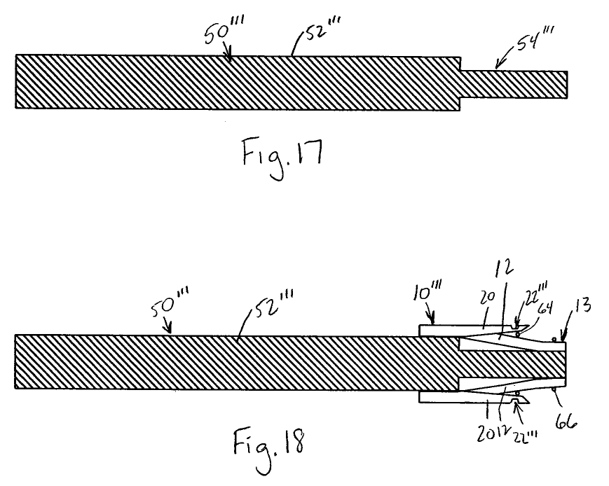

Referring to FIGS. 17 and 18, a method of manufacturing the stent 10′″ of FIG. 16 will be described. A grinding rod 50′″ has a generally cylindrical body 52′″ with a circumferential recess 54′″ formed at the complete end of the rod 50′″. The circumferential recess 54′″ is configured to receive the struts 12 and the end 13 of the stent 10′″ below the surface of the barbs 20. To ensure the barbs 20 do not deflect inward, a support wire 64 is positioned between the barbs 20 and the struts 12. The support wire 64 maintains the barbs 20 in the grinding plane such that belt grooves 22′″ may be formed therein. A retaining wire 66 may be provided about the end 13 of the stent 10′″ to ensure it is maintained away from the grinding plane.

What is claimed:

1. A stent assembly comprising:
a stent body;
at least one barb extending from the stent body and configured such that a free end thereof is biased to extend radially outward from the stent body; and
a belt releasably positioned about the stent body and aligned with the barb to constrain the barb to a position with the free end proximate to the stent body.

2. The stent according to claim 1 wherein the stent body comprises a plurality of axially extending struts.

3. The stent according to claim 1 wherein the stent body comprises a lattice structure.

4. The stent according to claim 1 wherein the stent body comprises a helical structure.

5. The stent according to claim 1 wherein the at least one barb is formed integrally with the stent body.

6. The stent according to claim 1 wherein the barb free end has a pointed tip.

7. The stent according to claim 6 wherein the pointed tip converges radially outward.

8. The stent according to claim 6 wherein the pointed tip converges radially inward.

9. The stent according to claim 6 wherein a circumferential groove configured to receive the belt extends across the pointed tip.

10. The stent according to claim 9 wherein the circumferential groove does not extend across the stent body.

11. The stent according to claim 1 wherein a circumferential groove extends about the stent body and is configured to receive the belt.

12. The stent according to claim 11 wherein the circumferential groove extends across a portion of the at least one barb.

13. The stent according to claim 1 wherein a release wire releasably secures the belt.

14. The stent according to claim 1 wherein at least one secondary belt radially constrains the stent body.

15. The stent according to claim 14 wherein a single release wire releasably secures the belt and the at least one secondary belt.

16. The stent according to claim 14 wherein a first release wire releasably secures the belt and a second release wire releasably secures the at least one secondary belt.

17. A method of forming a stent assembly, comprising:
forming a stent body having at least one barb with a free end extending radially outward from the stent body; and
releasably securing a belt about the stent body in alignment with the barb to constrain the barb to a position with the free end proximate to the stent body.

18. The method according to claim 17 further comprising:
defining a circumferential groove about the stent body configured to receive the belt.

19. The method according to claim 18 wherein the step of defining the circumferential groove includes deflecting the at least one barb radially inward such that the at least one barb does not include the circumferential groove.

20. The method according to claim 17 further comprising:
defining a circumferential groove across the at least one barb configured to receive the belt.

21. The method according to claim 20 wherein the step of defining the circumferential groove includes deflecting the stent body radially inward such that the stent does not include the circumferential groove.
